(19) United States
(12) Patent Application Publication
(10) Pub. No.: US 2014/0364742 A1
(43) Pub. Date: Dec. 11, 2014

(54) ULTRASONIC PROBE AND MANUFACTURING METHOD THEREOF

(71) Applicant: SAMSUNG ELECTRONICS CO., LTD., Suwon-si (KR)

(72) Inventors: Kyung Il CHO, Seoul (KR); Bae Hyung KIM, Yongin-si (KR); Young Il KIM, Suwon-si (KR); Jong Keun SONG, Yongin-si (KR); Seung Heun LEE, Seoul (KR)

(73) Assignee: SAMSUNG ELECTRONICS CO., LTD., Suwon-si (KR)

(21) Appl. No.: 14/301,741

(22) Filed: Jun. 11, 2014

(30) Foreign Application Priority Data
Jun. 11, 2013 (KR) 10-2013-0066303

(51) Int. Cl. H05K 7/20; A61B 8/00

(52) U.S. Cl. CPC H05K 7/2039; A61B 8/4444; H05K 2007/20527
USPC 600/459; 29/594

(57) ABSTRACT

Disclosed herein is an ultrasonic probe capable of emitting heat generated by a transducer outside the ultrasonic probe using a heat radiation plate. The ultrasonic probe includes a transducer configured to generate an ultrasonic wave, a heat spreader provided on a surface of the transducer, the heat spreader being configured to absorb heat generated by the transducer, at least one heat radiation plate which contacts at least one side of the heat spreader, and at least one board installed on the at least one heat radiation plate so as to transfer heat generated by the at least one board to the at least one heat radiation plate.

### CROSS-REFERENCE TO RELATED APPLICATION

This application claims the benefit of Korean Patent Application No. 2013-0066303, filed on Jun. 11, 2013 in the Korean Intellectual Property Office, the disclosure of which is incorporated herein by reference in its entirety.

### BACKGROUND

1. Field

Exemplary embodiments of the present disclosure relate to an ultrasonic probe of an ultrasonic diagnostic apparatus to diagnose diseases.

2.Description of the Related Art

An ultrasonic diagnostic apparatus is an apparatus which projects ultrasonic waves from a surface of an object toward a target part inside the object and receives an ultrasonic echo signal reflected therefrom in order to noninvasively obtain a monolayer of soft tissue or an image related to a blood stream.

The ultrasonic diagnostic apparatus may be small and cheap, and may display diagnostic imaging in real time, compared to other imaging diagnostic devices such as an X-ray device, a CT scanner (computerized tomography scanner), and a nuclear medicine diagnostic device. In addition, since the ultrasonic diagnostic apparatus does not cause radiation exposure, the ultrasonic diagnostic apparatus may be inherently safe. Accordingly, the ultrasonic diagnostic apparatus is widely utilized for cardiac, abdominal, and urologic diagnosis as well as maternity diagnosis.

The ultrasonic diagnostic apparatus include an ultrasonic probe which projects ultrasonic waves onto an object and receives ultrasonic echo signals reflected from the object in order to image the interior of the object.

In general, a piezoelectric substance, which converts electric energy into mechanical vibration energy to generate an ultrasonic wave, is widely used as a transducer which generates an ultrasonic wave in the ultrasonic probe.

A capacitive micromachined ultrasonic transducer (hereinafter, also referred to as “cMUT”), which is a transducer based upon novel concepts, has recently been developed.

Recently, research and development of a two-dimensional (2D) array transducer has been actively performed, and the cMUT is well suited to be applied to 2D array transducers, thereby facilitating development of a multichannel transducer.

On the other hand, in a transducer having a small number of channels, a heating value of about 1 W is generated by an electric circuit or the like to drive the probe, and such a heating value may be naturally emitted through a probe casing. However, in a transducer having a large number of channels, an increased heating value of about 7 W is generated, and thus, technologies to radiate and cool the ultrasonic probe are needed.

### SUMMARY

Therefore, it is an aspect of the exemplary embodiments to provide an ultrasonic probe capable of emitting heat generated by a transducer outside the ultrasonic probe using a heat radiation plate.

Additional aspects of the exemplary embodiments will be set forth in part in the description which follows and, in part, will be obvious from the description, or may be learned by practice of the exemplary embodiments.

In accordance with an aspect of an exemplary embodiment, there is provided an ultrasonic probe including a transducer configured to generate an ultrasonic wave, a heat spreader provided on a surface of the transducer, the heat spreader being configured to absorb heat generated by the transducer, at least one heat radiation plate which contacts at least one side of the heat spreader, and at least one board installed on the at least one heat radiation plate so as to transfer heat generated by the at least one board to the at least one heat radiation plate.

In accordance with another aspect of an exemplary embodiment, there is provided a method of manufacturing an ultrasonic probe, the method including providing a heat spreader on a surface of a transducer, the heat spreader being configured so as to absorb heat generated by the transducer, providing at least one heat radiation plate such that the at least one heat radiation plate contacts at least one side of the heat spreader, and installing at least one board on the at least one heat radiation plate so as to transfer heat generated by the at least one board to the at least one heat radiation plate.

### BRIEF DESCRIPTION OF THE DRAWINGS

These and/or other aspects of the exemplary embodiments will become apparent and more readily appreciated from the following description of the exemplary embodiments, taken in conjunction with the accompanying drawings of which:

FIG. 1 is a perspective view illustrating an external appearance of an ultrasonic probe according to an exemplary embodiment;

FIG. 2 is a perspective view illustrating a structure of the ultrasonic probe of FIG. 1 with the housing removed;

FIG. 3 is a perspective view illustrating a structure in which a heat pipe is installed on a heat spreader;

FIG. 4 is a perspective view illustrating an external appearance of a rear housing of the ultrasonic probe in FIG. 1;

FIG. 5 is a cross-sectional view taken along direction A-A′ in FIG. 4;

FIG. 6 is an exploded perspective view illustrating the ultrasonic probe in FIG. 1;

FIG. 7 is a view illustrating an operation principle of the heat pipe; and

FIGS. 8, 9, 10 and 11 are views illustrating a process of manufacturing the ultrasonic probe according to an exemplary embodiment.

### DETAILED DESCRIPTION

Reference will now be made in detail to the exemplary embodiments of the present disclosure, examples of which are illustrated in the accompanying drawings, wherein like reference numerals refer to like elements throughout.

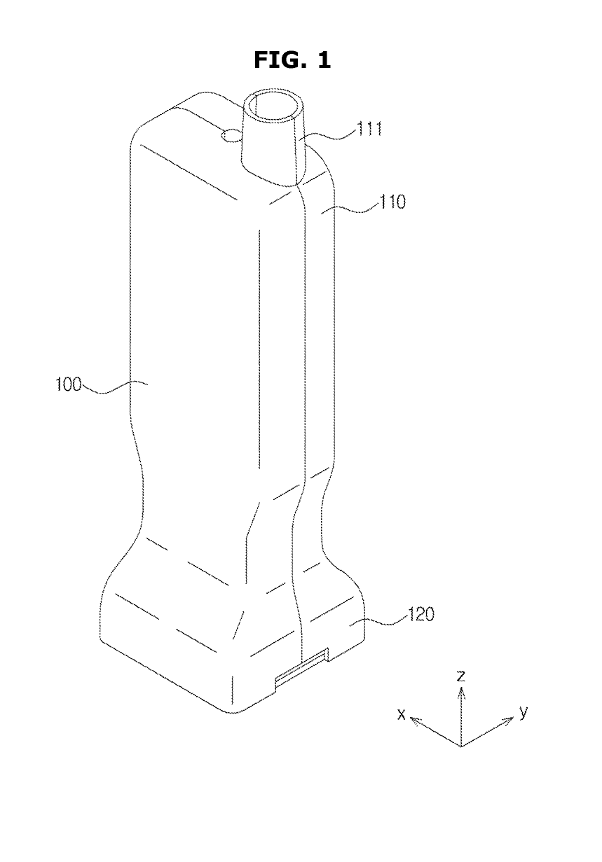

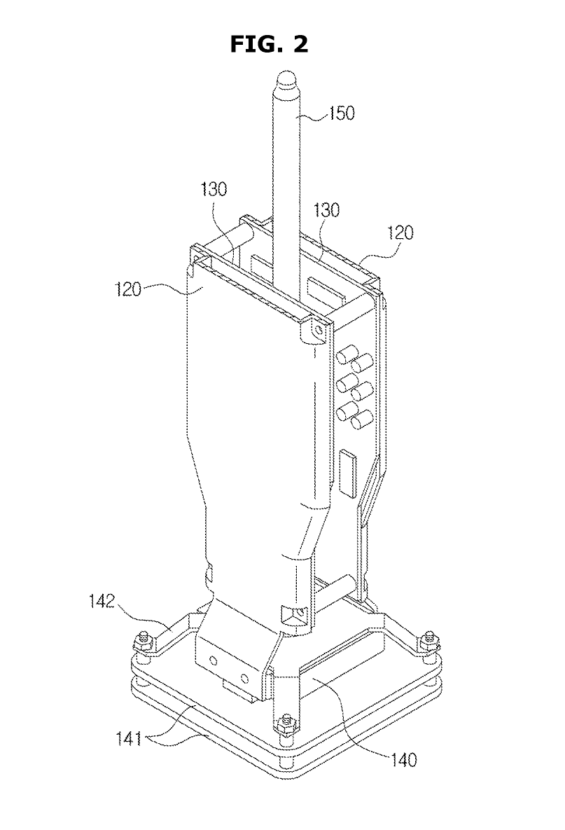

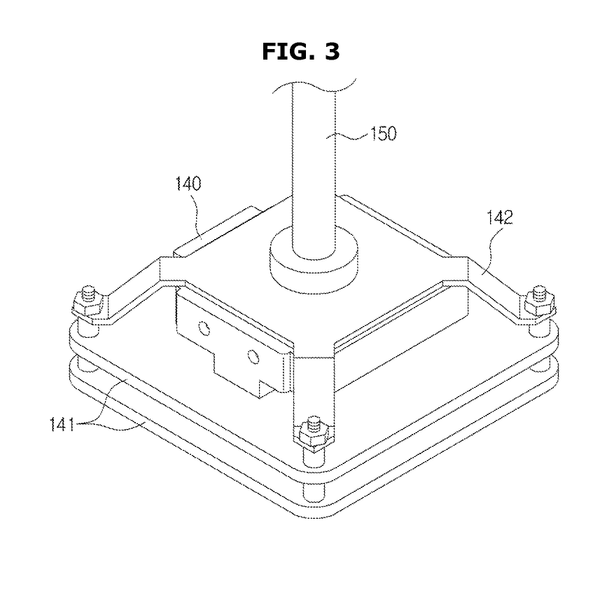

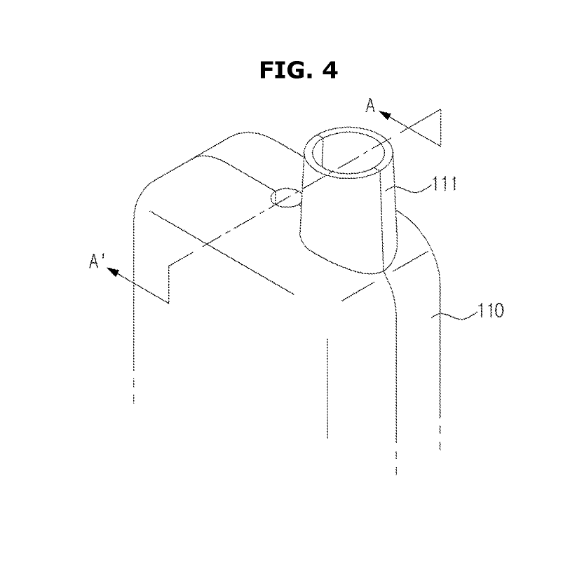

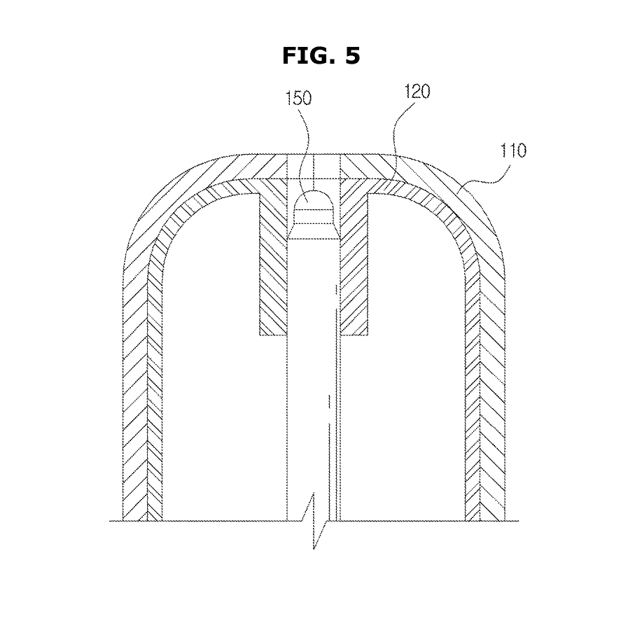

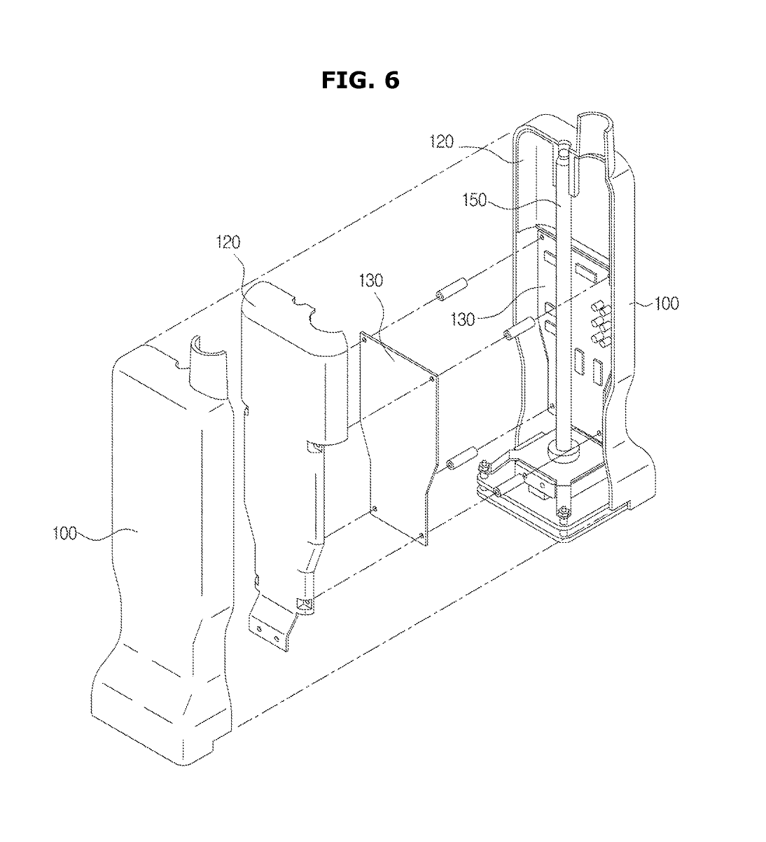

FIG. 1 is a perspective view illustrating an external appearance of an ultrasonic probe according to an exemplary embodiment. FIG. 2 is a perspective view illustrating a structure of the ultrasonic probe of FIG. 1, from which the housing 100 is removed. FIG. 3 is a perspective view illustrating a structure in which a heat pipe 150 is installed on a heat spreader 140. FIG. 4 is a perspective view illustrating an external appearance of a rear housing 110 of the ultrasonic probe in FIG. 1. FIG. 5 is a cross-sectional view taken along direction A-A′ in FIG. 4. FIG. 6 is an exploded perspective view illustrating the ultrasonic probe in FIG. 1.

Referring to FIGS. 1 to 6 and FIG. 8, the ultrasonic probe includes a transducer 101, a heat spreader 140 to absorb heat generated by the transducer 101, a heat pipe 150 to transfer heat absorbed by the heat spreader 140, heat radiation plates 120 installed on side surfaces of the heat spreader 140, boards 130 installed on inner sides of the respective heat radiation plates 120, and a housing 100 defining an external appearance of the ultrasonic probe.

According to an exemplary embodiment, a magnetostrictive ultrasonic transducer using a magnetostrictive effect of a magnetic substance which is mainly used in the ultrasonic probe apparatus, a piezoelectric ultrasonic transducer using a piezoelectric effect of a piezoelectric substance, or the like may be utilized as the ultrasonic transducer 101. In addition, according to an exemplary embodiment, a capacitive micromachined ultrasonic transducer (hereinafter, referred to as “cMUT”) which transmits and receives ultrasonic waves using vibrations of several hundred or thousands of micromachined thin films may also be utilized as the ultrasonic transducer 101.

The heat spreader 140 absorbs heat generated by the transducer 101 and is installed on a rear surface of the transducer 101. The heat spreader 140 may be made of a metal such as aluminum. The heat spreader 140 comes into thermal contact with the transducer 101 to absorb heat generated by the transducer 101. FIG. 3 shows a structure of the heat spreader 140 in a case in which the cMUT is used as an example of the transducer 101. In general, a cMUT array is bonded to an integrated circuit such as an ASIC (application specific integrated circuit) in a flip chip bonding manner, and signal lines of the ASIC to which the cMUT array is bonded may be bonded onto a printed circuit board 141 in a wire bonding manner. FIG. 3 shows a state in which the heat spreader 140 is installed on the printed circuit board 141. The heat spreader 140 is installed by being inserted into the printed circuit board 141 to come into thermal contact with the transducer 101.

The heat spreader 140 is provided, on a rear surface thereof, with a fixing plate 142 to fix the heat spreader 140 to the printed circuit board 141.

The heat spreader 140 may be provided such that the heat spreader 140 comes into direct contact with the transducer 101 or a predetermined gap is defined between the heat spreader 140 and the transducer 101 without direct contact therebetween. The gap between the heat spreader 140 and the transducer 101 may be filled with thermal grease or a phase change material which is a thermal medium having good thermal conductivity. Heat generated by the transducer 101 is directly transferred through the heat spreader 140, or is transferred to the heat spreader 140 through the thermal grease or the phase change material filled in the gap.

The heat spreader 140 may be provided with the heat pipe 150 to transfer heat absorbed by the heat spreader 140 in a direction opposite to a direction in which ultrasonic waves are projected, namely, in a z-axis direction.

The heat spreader 140 may be provided with an insertion groove into which the heat pipe 150 may be inserted, and the heat pipe 150 may be inserted into the insertion groove to be installed on the heat spreader 140. In order to efficiently transfer heat from the heat spreader 140 to the heat pipe 150, the insertion groove provided in the heat spreader 140 may have a depth which reaches a thermal contact surface between the heat spreader 140 and the transducer 101. In other words, the heat pipe 150 may be inserted to such a degree as to reach the thermal contact surface between the heat spreader 140 and the transducer 101.

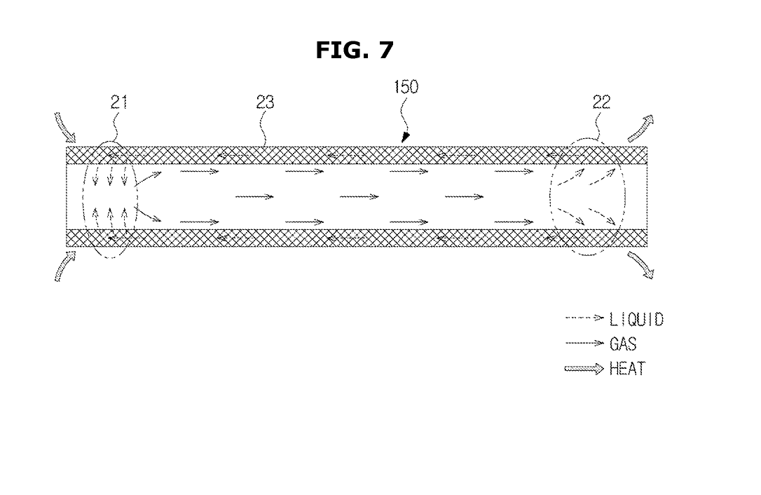

FIG. 7 is a view illustrating an operation principle of the heat pipe 150.

The heat pipe 150 is a device, evacuated to a vacuum state, in which a working fluid is injected into a closed pipe-shaped container.

The working fluid in the heat pipe 150 is present in two phases to transfer heat.

Referring to FIG. 7, when heat is applied to an evaporation portion 21 of the heat pipe 150, the heat is transferred into the heat pipe 150 by a thermal conductivity via an outer wall.

In the inside of the heat pipe 150 having high pressure, even low temperatures may cause evaporation of the working fluid to occur on a surface of a wick 23.

Gas density and pressure are increased in the evaporation portion 21 due to the evaporation of the working fluid, and thus, a pressure gradient is formed in a gas passage of a central portion of the heat pipe 150 in a direction toward a condensation portion 22 having relatively low density of gas and pressure so as to move a gas.

In this case, the moving gas is moved in a state of having a large amount of heat of no less than evaporative latent heat.

The gas moved to the condensation portion 22 dissipates heat while condensing on an inner wall of the condensation portion 22 having a relatively low temperature, and returns back to a liquid phase.

The working fluid returned to the liquid phase is again moved toward the evaporation portion 21 through pores within the wick 23 by capillary pressure of the wick or gravity.

Repetition of these processes enables heat transfer to be consistently carried out.

The evaporation portion 21 of the heat pipe 150 is installed to come into contact with the heat spreader 140 which absorbs heat generated by the transducer 101, and the heat pipe 150 transfers the heat generated by the transducer 101 to the rear of the ultrasonic probe according to the above-mentioned heat transfer process. The condensation portion 22 of the heat pipe 150 is installed to come into thermal contact with the heat radiation plates 120 (see FIG. 6) which are described later, and thus may also transfer heat to the heat radiation plates 120.

FIG. 2 shows that the two heat radiation plates 120 having a shape corresponding to the housing 100 of FIG. 1 are installed on both sides of the heat spreader 140.

The heat radiation plates 120 may be installed on the heat spreader 140 through fastening members, and may emit heat absorbed by the heat spreader 140 into the air. The heat radiation plates 120 have a shape similar to a shape of the housing 100 shown in FIG. 1, so that when the housing 100 is installed outside the heat radiation plates 120, a space between each heat radiation plate 120 and the housing 100 may be minimized and heat radiation efficiency may be improved.

In addition, the two heat radiation plates 120 serve as frames to which the boards 130 vertically connected to the transducer 101 may be attached as shown in FIG. 2, in addition to having heat radiation functions. The heat radiation plates 120 may be made of metal having a high thermal conductivity, such as aluminum or copper.

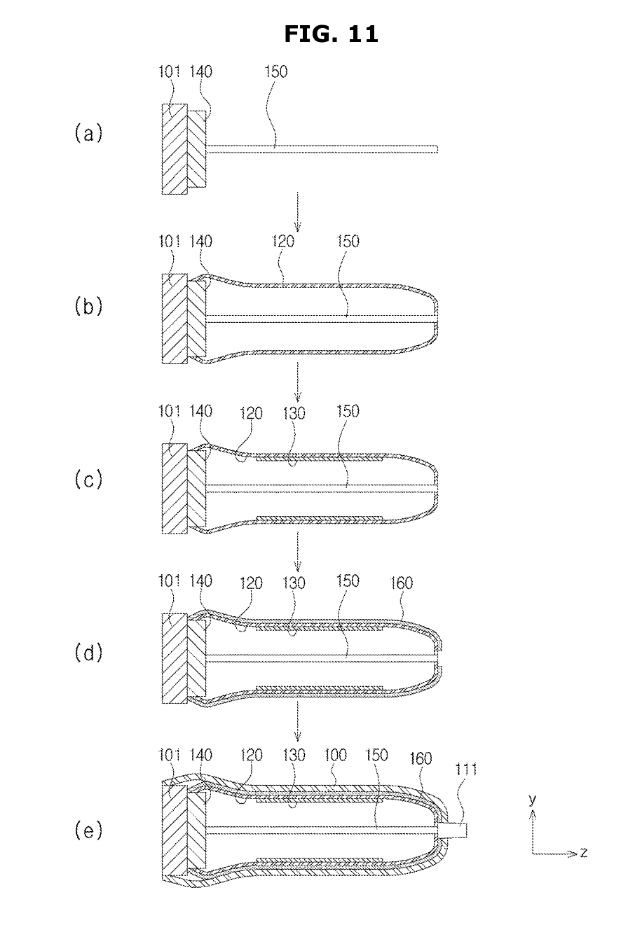

The spaces between the heat radiation plates 120 and the housing 100 may be further provided with heat radiation members 160 (see FIG. 11) made of graphite. That is, according to an exemplary embodiment, the two heat radiation members 160 having a shape similar to a shape of each of the heat radiation plates 120 and the housing 100 are respectively installed outside the two heat radiation plates 120, the housing 100 is installed outside the heat radiation members 160, and the heat radiation members 160 made of graphite may be installed in the respective spaces between the heat radiation plates 120 and the housing 100. Graphite is a material having a thermal conductivity more than two times a thermal conductivity of aluminum. The heat radiation members 160 are filled in the spaces between the heat radiation plates 120 and the housing 100, instead of filling the spaces with air, thereby enabling heat transfer and heat radiation to be more efficiently performed than when the heat radiation members 160 are not present.

The heat radiation members 160 may be installed to come into contact with a cable extension portion 111 of the rear housing 110 shown in FIG. 4. The cable extension portion 111 may be made of a material having a high thermal conductivity to emit heat transferred from the heat radiation members 160 to the outside.

Each of the boards 130 receives a signal related to driving of the ultrasonic probe through the cable extension portion 111 of the rear housing 110 from a cable connected to the inside of the ultrasonic probe so as to output a signal to control driving of the transducer 101.

The board 130 includes a circuit board on which chips to control driving of the ultrasonic probe are mounted.

The board 130 is electrically connected to the transducer 101 via a flexible printed circuit board or the like so as to output the signal to the transducer 101. The board 130 may be electrically connected to the circuit board to which the cMUT is mounted and the ASIC is bonded. As described above, the board 130 may be installed inside each heat radiation plate 120 so as to be fixed thereto.

The rear housing 110 is shown in FIG. 4, and is provided with the cable extension portion 111 as described above. The cable, which is electrically connected to the board 130 to output a control signal applied from the outside to the board 130, extends through the cable extension portion 111 of the rear housing 110. The cable extension portion 111 is made of a material having a high thermal conductivity, thereby enabling heat transferred from each heat radiation member 160 to be emitted to the outside.

FIG. 5 is a cross-sectional view cutting the rear housing 110 shown in FIG. 4 in direction A-A′. As shown in FIG. 5, each heat radiation plate 120 may be provided such that a rear end of the heat radiation plate 120 comes into contact with the heat pipe 150. As shown in FIG. 5, the condensation portion 22 of the heat pipe 150 comes into contact with the rear end of the heat radiation plate 120, so that heat absorbed by the heat spreader 140 may be transferred rearward of the ultrasonic probe along the heat pipe 150 to be emitted through the heat radiation plate 120 to the outside.

FIG. 6 is an exploded perspective view illustrating the ultrasonic probe in FIG. 1. As shown in FIG. 6, the ultrasonic probe includes the housing 100, the heat radiation plates 120 provided inside the housing 100, and the boards 130 provided inside the respective heat radiation plates 120. In addition, a front housing 120 is provided with an assembly of the heat spreader 140 and the heat pipe 150.

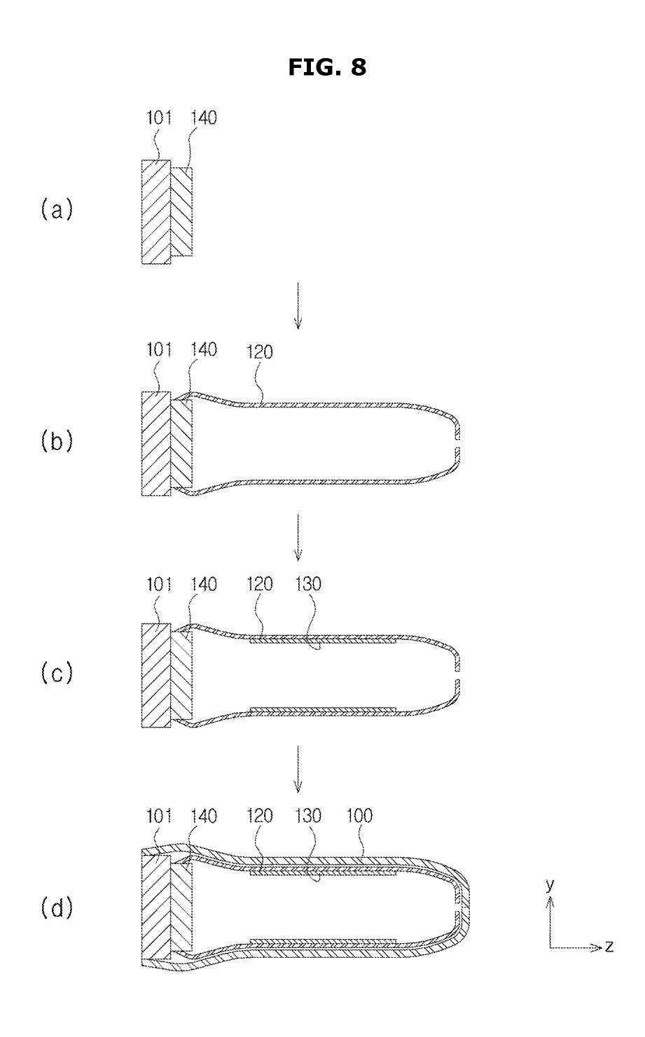

FIGS. 8 to 11 are views illustrating a process of manufacturing the ultrasonic probe. FIGS. 8 to 11 schematically show various configurations of the ultrasonic probe.

Referring to (a) of FIG. 8, the heat spreader 140 is installed on the rear surface of the transducer 101 in order to absorb heat generated by the transducer 101.

The installed heat spreader 140 may be made of a metal such as aluminum having a high thermal conductivity, and may come into direct contact with the transducer 101 or come into indirect contact with the transducer 101 with a thermal medium interposed therebetween, thereby enabling heat generated by the transducer 101 to be absorbed.

After the heat spreader 140 is installed, the heat radiation plates 120, which are supplied with heat absorbed by the heat spreader 140 to emit the heat to the outside, are installed to the heat spreader 140 (see (b) of FIG. 8).

The heat radiation plates 120 may also be made of a metal having a high thermal conductivity. The heat radiation plates 120 may be coupled to the side surfaces of the heat spreader 140 using the fastening members, or may be installed by being inserted into the heat spreader 140.

The heat radiation plates 120 may be previously manufactured so as to have a shape similar to a shape of the housing 100.

After the heat radiation plates 120 are installed, the boards 130 are installed inside of the respective heat radiation plates 120 (see (c) of FIG. 8).

The boards 130 may be installed inside of the respective heat radiation plates 120 by using the fastening members. Each of the boards 130 receives a signal related to driving of the ultrasonic probe through the cable extension portion 111 of the rear housing 110 from the cable connected to the inside of the ultrasonic probe so as to output a signal to control driving of the transducer 101. The board 130 includes the circuit board on which chips to control driving of the ultrasonic probe are mounted. The board 130 is electrically connected to the transducer 101 via the flexible printed circuit board or the like so as to output the signal to the transducer 101.

After the boards 130 are installed, the housing 100 is installed outside the heat radiation plates 120 (see (d) of FIG. 8).

The form of each heat radiation plate 120, for example, the bent form, has a shape corresponding to the housing 100. Thus, when the housing 100 is installed, the housing 100 may be pressed against the heat radiation plate 120, with the consequence that a gap between the housing 100 and the heat radiation plate 120 is very small. Therefore, heat radiation efficiency through the heat radiation plate 120 is not deteriorated. The space between the housing 100 and the heat radiation plate 120 may be determined such that radiation efficiency of heat emitted from the heat radiation plate 120 through the housing 100 to the outside reaches a certain level or more, as determined by an experiment.

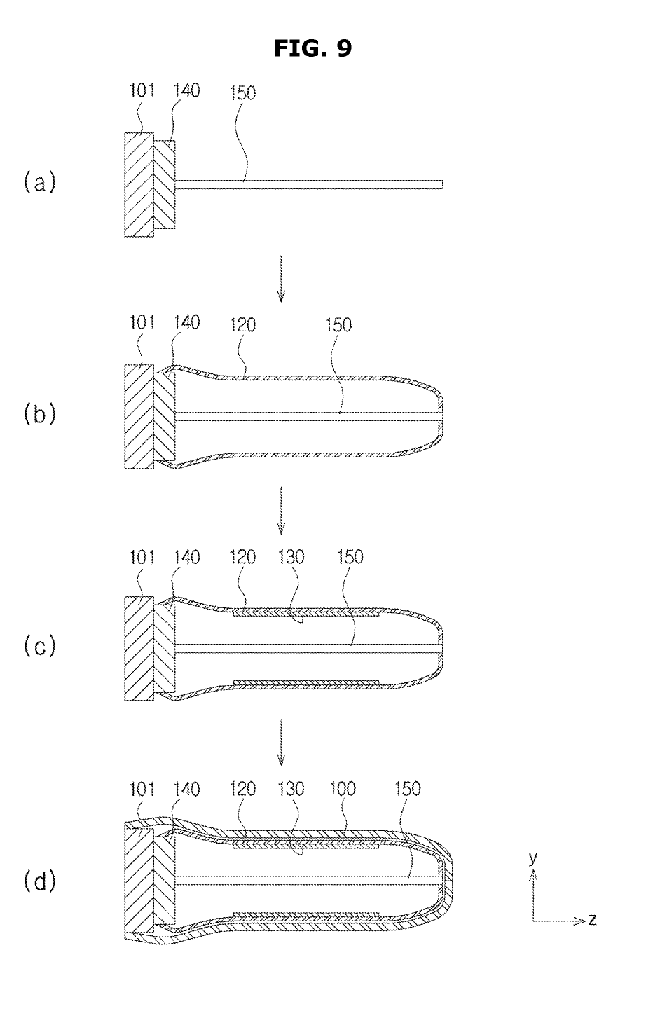

Referring to (a) of FIG. 9, the heat spreader 140 is installed on the rear surface of the transducer 101 in order to absorb heat generated by the transducer 101, and the heat pipe 150 is installed on the rear surface of the heat spreader 140.

The installed heat spreader 140 may be made of a metal having a high thermal conductivity, such as aluminum, and may come into direct contact with the transducer 101 or come into indirect contact with the transducer 101 with a thermal medium interposed therebetween, thereby enabling heat generated by the transducer 101 to be absorbed.

The heat spreader 140 may be provided with the heat pipe 150 to transfer heat absorbed by the heat spreader 140 in a direction opposite to a direction in which ultrasonic waves are projected, namely, in a z-axis direction.

The heat spreader 140 may be provided with an insertion groove into which the heat pipe 150 may be inserted, and the heat pipe 150 may be inserted into the insertion groove to be installed on the heat spreader 140. In order to efficiently transfer heat from the heat spreader 140 to the heat pipe 150, the insertion groove provided in the heat spreader 140 may have a depth which reaches a thermal contact surface between the heat spreader 140 and the transducer 101. In other words, the heat pipe 150 may be inserted to such a degree as to reach the thermal contact surface between the heat spreader 140 and the transducer 101.

After the heat spreader 140 and the heat pipe 150 are installed, the heat radiation plates 120 to emit heat absorbed by the heat spreader 140 and heat transferred through the heat pipe 150 to the outside are installed on the heat spreader 140 (see (b) of FIG. 9).

The heat radiation plates 120 may be made of a metal having a high thermal conductivity. The heat radiation plates 120 may be coupled to the side surfaces of the heat spreader 140 through the fastening members, or may be installed by being inserted into the heat spreader 140. In addition, the heat radiation plates 120 may be previously manufactured so as to have a shape similar to a shape of the housing 100. As shown in FIG. 9, the rear ends of the heat radiation plates 120 are provided so as to come into thermal contact with the condensation portion 22 of the heat pipe 150. Accordingly, the heat radiation plates 120 emit heat absorbed by the heat spreader 140 and heat transferred through the heat pipe 150 to the outside.

After the heat radiation plates 120 are installed, the boards 130 are installed inside of the respective heat radiation plates 120 (see (c) of FIG. 9).

The boards 130 may be installed inside of the respective heat radiation plates 120 by the fastening members. Each of the boards 130 receives a signal related to driving of the ultrasonic probe through the cable extension portion 111 of the rear housing 110 from the cable connected to the inside of the ultrasonic probe so as to output a signal to control driving of the transducer 101. The board 130 includes the circuit board on which chips to control driving of the ultrasonic probe are mounted. The board 130 is electrically connected to the transducer 101 via the flexible printed circuit board or the like so as to output the signal to the transducer 101.

After the boards 130 are installed, the housing 100 is installed outside the heat radiation plates 120 (see (d) of FIG. 9).

The form of each heat radiation plate 120, for example, the bent form, has a shape corresponding to the housing 100. Thus, when the housing 100 is installed, the housing 100 may be pressed against the heat radiation plate 120, with the consequence that a gap between the housing 100 and the heat radiation plate 120 is very small. Therefore, heat radiation efficiency through the heat radiation plate 120 is not deteriorated. The space between the housing 100 and the heat radiation plate 120 may be determined such that radiation efficiency of heat emitted from the heat radiation plate 120 through the housing 100 to the outside reaches a certain level or more, as determined by an experiment.

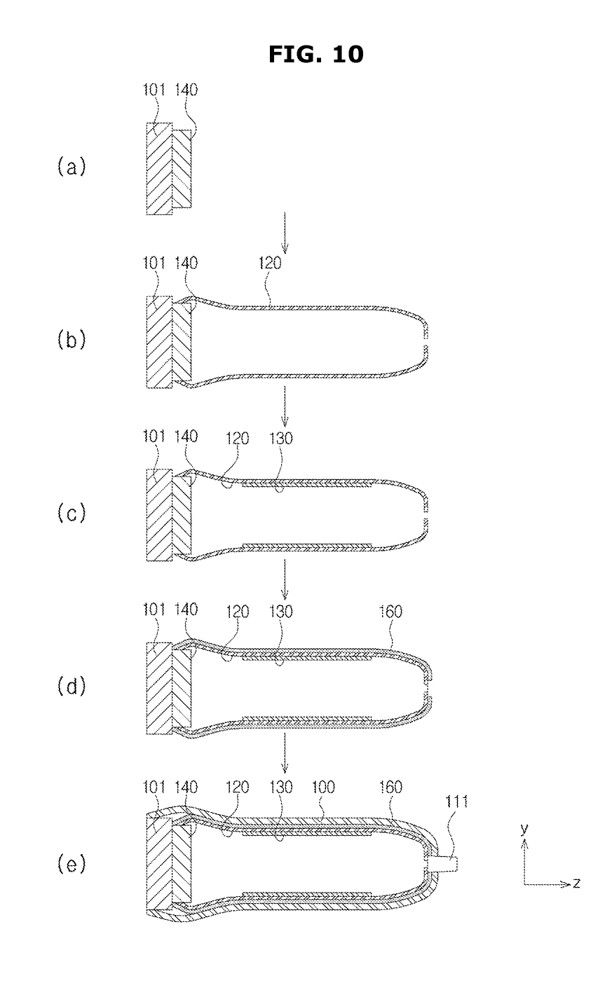

Referring to (a) of FIG. 10, the heat spreader 140 is installed on the rear surface of the transducer 101 in order to absorb heat generated by the transducer 101.

The installed heat spreader 140 may be made of a metal having a high thermal conductivity, such as aluminum, and may come into direct contact with the transducer 101 or come into indirect contact with the transducer 101 with a thermal medium interposed therebetween, thereby enabling heat generated by the transducer 101 to be absorbed.

After the heat spreader 140 is installed, the heat radiation plates 120, which are supplied with heat absorbed by the heat spreader 140 to emit the heat to the outside, are installed on the heat spreader 140 (see (b) of FIG. 10).

The heat radiation plates 120 may also be made of a metal having a high thermal conductivity. The heat radiation plates 120 may be coupled to the side surfaces of the heat spreader 140 by using the fastening members, or may be installed by being inserted into the heat spreader 140.

The heat radiation plates 120 may be previously manufactured so as to have a shape similar to a shape of the housing 100.

After the heat radiation plates 120 are installed, the boards 130 are installed inside of the respective heat radiation plates 120 (see (c) of FIG. 10).

The boards 130 may be installed inside of the respective heat radiation plates 120 by the fastening members. Each of the boards 130 receives a signal related to driving of the ultrasonic probe through the cable extension portion 111 of the rear housing 110 from the cable connected to the inside of the ultrasonic probe so as to output a signal to control driving of the transducer 101. The board 130 includes the circuit board on which chips to control driving of the ultrasonic probe are mounted. The board 130 is electrically connected to the transducer 101 via the flexible printed circuit board or the like so as to output the signal to the transducer 101.

After the boards 130 are installed, heat radiation members 160, which may, for example, made of graphite, are installed outside the respective heat radiation plates 120 (see (d) of FIG. 10).

The two heat radiation members 160 having a shape similar to that of each of the heat radiation plates 120 and the housing 100 are respectively installed outside the two heat radiation plates 120, the housing 100 is installed outside the heat radiation members 160, and the heat radiation members 160 made of graphite are installed in the respective spaces between the heat radiation plates 120 and the housing 100. Graphite is a material having a thermal conductivity more than two times a thermal conductivity of aluminum. The heat radiation members 160 are filled in the spaces between the heat radiation plates 120 and the housing 100, instead of filling the spaces with air, thereby enabling heat transfer and heat radiation to be more efficiently performed than when the heat radiation members 160 are not present.

After the heat radiation members 160 made of graphite are installed, the housing 100 is installed outside the heat radiation members 160 (see (e) of FIG. 10).

The form of each heat radiation plate 120, for example, the bent form, has a shape corresponding to the housing 100. Thus, when the housing 100 is installed, the housing 100 may be pressed against the heat radiation plate 120, with the consequence that a gap between the housing 100 and the heat radiation plate 120 is very small. Therefore, heat radiation efficiency through the heat radiation plate 120 is not deteriorated. The space between the housing 100 and the heat radiation plate 120 may be determined such that radiation efficiency of heat emitted from the heat radiation plate 120 through the housing 100 to the outside reaches a certain level or more, as determined by an experiment. In addition, the cable extension portion 111 provided in the rear end of the housing 100 is provided so as to come into thermal contact with the heat radiation members 160 which may be made of graphite. The cable extension portion 111 may be made of a material having a high thermal conductivity to emit heat transferred from the heat radiation members 160 to the outside.

Referring to (a) of FIG. 11, the heat spreader 140 is installed on the rear surface of the transducer 101 in order to absorb heat generated by the transducer 101, and the heat pipe 150 is installed on the rear surface of the heat spreader 140.

The installed heat spreader 140 may be made of a metal having a high thermal conductivity, such as aluminum, and may come into direct contact with the transducer 101 or come into indirect contact with the transducer 101 by interposing a thermal medium therebetween, thereby enabling heat generated by the transducer 101 to be absorbed.

The heat spreader 140 may be provided with the heat pipe 150 to transfer heat absorbed by the heat spreader 140 in a direction opposite to a direction in which ultrasonic waves are projected, namely, in a z-axis direction.

The heat spreader 140 may be provided with the insertion groove into which the heat pipe 150 may be inserted, and the heat pipe 150 may be inserted into the insertion groove to be installed on the heat spreader 140. In order to efficiently transfer heat from the heat spreader 140 to the heat pipe 150, the insertion groove provided in the heat spreader 140 may have a depth which reaches a thermal contact surface between the heat spreader 140 and the transducer 101. In other words, the heat pipe 150 may be inserted to such a degree as to reach the thermal contact surface between the heat spreader 140 and the transducer 101.

After the heat spreader 140 and the heat pipe 150 are installed, the heat radiation plates 140 to emit heat absorbed by the heat spreader 140 and heat transferred through the heat pipe 150 to the outside are installed on the heat spreader 140 (see (b) of FIG. 11).

The heat radiation plates 120 may also be made of a metal having a high thermal conductivity. The heat radiation plates 120 may be coupled to the side surfaces of the heat spreader 140 by the fastening members, or may be installed by being inserted into the heat spreader 140.

The heat radiation plates 120 may be previously manufactured so as to have a shape similar to a shape of the housing 100.

After the heat radiation plates 120 are installed, the boards 130 are installed inside of the respective heat radiation plates 120 (see (c) of FIG. 11).

The boards 130 may be installed inside of the respective heat radiation plates 120 by the fastening members. Each of the boards 130 receives a signal related to driving of the ultrasonic probe through the cable extension portion 111 of the rear housing 110 from the cable connected to the inside of the ultrasonic probe so as to output a signal to control driving of the transducer 101. The board 130 includes the circuit board on which chips to control driving of the ultrasonic probe are mounted. The board 130 is electrically connected to the transducer 101 via the flexible printed circuit board or the like so as to output the signal to the transducer 101.

After the boards 130 are installed, the heat radiation members 160 which may be made of graphite are installed outside the respective heat radiation plates 120 (see (d) of FIG. 11).

The two heat radiation members 160 having a shape similar to a shape of each of the heat radiation plates 120 and the housing 100 are respectively installed outside the two heat radiation plates 120, the housing 100 is installed outside the heat radiation members 160, and the heat radiation members 160 which may be made of graphite are thus installed in the respective spaces between the heat radiation plates 120 and the housing 100. Graphite is a material having a thermal conductivity more than two times a thermal conductivity of aluminum. The heat radiation members 160 are filled, in the spaces between the heat radiation plates 120 and the housing 100, instead of filling the spaces with air, thereby enabling heat transfer and heat radiation to be more efficiently performed than when the heat radiation members 160 are not present.

After the heat radiation members 160 made of graphite are installed, the housing 100 is installed outside the heat radiation members 160 (see (e) of FIG. 11).

The form of each heat radiation plate 120, for example, the bent form, has a shape corresponding to the housing 100. Thus, when the housing 100 is installed, the housing 100 may be pressed against the heat radiation plate 120, with the consequence that a gap between the housing 100 and the heat radiation plate 120 is very small. Therefore, heat radiation efficiency through the heat radiation plate 120 is not deteriorated. The space between the housing 100 and the heat radiation plate 120 may be determined such that radiation efficiency of heat emitted from the heat radiation plate 120 through the housing 100 to the outside reaches a certain level or more, as determined by an experiment. In addition, the cable extension portion 111 provided in the rear end of the housing 100 is provided so as to come into thermal contact with the heat radiation members 160 which may be made of graphite. The cable extension portion 111 may be made of a material having a high thermal conductivity to emit heat transferred from the heat radiation members 160 to the outside.

As is apparent from the above description, the exemplary embodiments may enhance thermal stability of an ultrasonic probe by efficiently emitting heat generated by the ultrasonic probe to the outside.

Although a few exemplary embodiments have been shown and described, it would be appreciated by those skilled in the art that changes may be made in these exemplary embodiments without departing from the principles and spirit of the exemplary embodiments, the scope of which is defined in the claims and their equivalents.

What is claimed is:

1. An ultrasonic probe comprising:
a transducer configured to generate an ultrasonic wave;
a heat spreader provided on a surface of the transducer, the heat spreader being configured to absorb heat generated by the transducer;
at least one heat radiation plate which contacts at least one side of the heat spreader; and
at least one board installed on the at least one heat radiation plate so as to transfer heat generated by the at least one board to the at least one heat radiation plate.

2. The ultrasonic probe according to claim 1, further comprising a housing which houses the transducer, the heat spreader, the at least one heat radiation plate, and the at least one board,
wherein the heat radiation plate has a shape corresponding to a shape of the housing and is configured to emit heat absorbed by the heat spreader.

3. The ultrasonic probe according to claim 2, wherein a space between the housing and the heat radiation plate is smaller than a predetermined gap.

4. The ultrasonic probe according to claim 2, further comprising a heat radiation member comprising graphite provided in a space between the housing and the heat radiation plate.

5. The ultrasonic probe according to claim 4, wherein the heat radiation member made of graphite has a shape corresponding to the shape of the housing.

6. The ultrasonic probe according to claim 4, further comprising:
a cable which is electrically connected to the board and configured to output a control signal transmitted from the outside to the board; and
a cable extension portion comprising a thermally conductive material, the cable extension portion being provided at an end of the housing such that the cable extends outward of the housing through the cable extension portion,
wherein the heat radiation member thermally contacts the cable extension portion to thereby emit heat through the cable extension portion.

7. The ultrasonic probe according to claim 1, further comprising a heat pipe installed on the heat spreader, the heat pipe being configured to transfer the heat absorbed by the heat spreader in a direction opposite to a direction in which the ultrasonic wave is projected.

8. The ultrasonic probe according to claim 7, wherein the heat pipe thermally contacts an end of the at least one heat radiation plate.

9. A method of manufacturing an ultrasonic probe comprising:
providing a heat spreader on a surface of a transducer, the heat spreader being configured to absorb heat generated by the transducer;
providing at least one heat radiation plate such that the at least one heat radiation plate contacts at least one side of the heat spreader; and
installing at least one board on the at least one heat radiation plate so as to transfer heat generated by the at least one board to the at least one heat radiation plate.

10. The method according to claim 9, further comprising installing a housing outside the heat radiation plate,
wherein the heat radiation plate has a shape corresponding to a shape of the housing and is configured to emit heat absorbed by the heat spreader.

11. The method according to claim 10, wherein a space between the housing and the heat radiation plate is smaller than a predetermined gap.

12. The method according to claim 9, further comprising installing a heat radiation member made of graphite between the housing and the heat radiation member.

13. The method according to claim 12, wherein the heat radiation member has a shape corresponding to the shape of the housing.

14. The method according to claim 12, wherein:
the housing comprises a cable extension portion comprising a thermally conductive material provided at a rear end of the housing, such that a cable configured to output a control signal applied from the outside to the board extends outward of the housing through the cable extension portion; and
the heat radiation member thermally contacts the cable extension portion to thereby emit absorbed heat through the cable extension portion.

15. The method according to claim 9, further comprising installing a heat pipe, which is configured to transfer heat absorbed by the heat spreader in a direction opposite to a direction in which an ultrasonic wave is projected, to the heat spreader.

16. The method according to claim 15, wherein the heat pipe thermally contacts a rear of the at least one heat radiation plate.

17. An ultrasonic probing apparatus, comprising:
a housing;
a transducer provided inside the housing at a first end of the housing, the transducer being configured to generate an ultrasonic wave; and
a heat pipe provided inside the housing and configured to transfer heat generated by the transducer to a second end of the housing opposite the first end of the housing.

18. The ultrasonic probing apparatus according to claim 17, further comprising a heat spreader provided between the transducer and the heat pipe, the heat spreader being configured to absorb the heat generated by the transducer and transfer the heat generated by the transducer to the heat pipe.

19. The ultrasonic probing apparatus according to claim 18, further comprising heat radiation plates provided inside of the housing and configured to transfer the heat generated by the transducer to the outside.

20. The ultrasonic probing apparatus according to claim 19, wherein the heat radiation plates have a same shape as a shape of the housing and are bent.
